(19) United States
(12) Patent Application Publication
(10) Pub. No.: US 2025/0197664 A1
(43) Pub. Date: Jun. 19, 2025

(54) ADHERENCE ENHANCING COATING FOR PLASTIC SURFACES

(71) Applicant: GRADIENTECH AB, Uppsala (SE)

(72) Inventor: Tobias KAMF, Björklinge (SE)

(21) Appl. No.: 18/849,074

(22) PCT Filed: Mar. 20, 2023
(86) PCT No.: PCT/SE2023/050238

§ 371 Date: Sep. 20, 2024

(30) Foreign Application Priority Data
Mar. 28, 2022 (SE) 2250374-2

(51) Int. Cl. C09D 105/00; C08J 7/04; C08J 7/043; C09D 5/00; C09D 7/61; C12M 1/12

(52) U.S. Cl. CPC C09D 105/00; C08J 7/042; C08J 7/043; C09D 5/002; C09D 7/61; C12M 25/04; C08J 2333/12; C08J 2405/00

(57) ABSTRACT

A surface coating method comprises heating a coating solution comprising a gelling agent, at least one sulfate salt and water to a temperature above the melting temperature of the gelling agent but below 100 #C. The heated coating solution is applied onto a plastic surface (12). The coating solution is dried on the plastic surface (12) to obtain a pre-coated plastic surface, onto which a liquid hydrogel is applied. The liquid hydrogel is solidified to form a solid hydrogel (30) adhering to the pre-coated plastic surface. The surface coating (20) enhances the adherence between the solid hydrogel (30) and the plastic surface (12).

### TECHNICAL FIELD

The present invention generally relates to enhancing adherence between hydrogels and plastic surfaces, and in particular to a coating solution for coating plastic surfaces to enhance adherence between hydrogels and the coated plastic surface.

### BACKGROUND

Hydrogels are widely used in various biotechnological applications. As an example, agar, which is a mixture of the linear polysaccharide agarose and a heterogeneous mixture of smaller molecules denoted agaropectin, is often used as a growth medium in agar plates or Petri dishes. Agar gels are porous and are therefore also used in monitoring microorganism motility and mobility. Correspondingly, agarose, the main component of agar, is the preferred gel matrix for work with proteins and nucleic acids since it has a broad range of physical, chemical and thermal stability. Hence, agarose is commonly used for electrophoretic separation of deoxyribonucleic acid (DNA) molecules in agarose gel electrophoresis but is also used in immunodiffusion and immunoelectrophoresis. Agarose gels are further used as gel matrix in protein purification as in gel filtration chromatography, affinity chromatography and ion exchange chromatography. Agarose is sometimes used instead of agar for culturing organisms since agar may contain impurities that can affect the growth of the organisms or some downstream procedure in the processing of the cultured organisms. In particular, agarose is often used as a support for three-dimensional (3D) culturing of cells or microorganisms and to monitor microorganism motility and mobility.

In several of these various biotechnological applications of hydrogels, such as agar or agarose, the hydrogel is applied onto a plastic surface and should adhere to this plastic surface. In some applications, a strong adhesion of the hydrogel to the plastic surface is required, for instance, if the hydrogel is subject to a pressure, such as caused by a fluid flow, that may detach the hydrogel from the plastic surface if the adhesion is not sufficiently strong.

One way of enhancing the adhesion between hydrogels and plastic surfaces is surface treatment or modification of the plastic surface including, for instance, oxygen plasma surface modification, and sand blasting. Also chemical modification by using an adhesion promoter has been suggested in DE 3032071, in which a lacquer-like water-insoluble coating is first applied to the plastic surface and can then react with hydroxyl groups of an agarose gel to permanently bind the agarose gel to the coating and thereby to the plastic surface.

These modifications of the plastic surface may, however, be undesired in some biotechnological applications as the surface modification may affect the biotechnological experiment to be conducted in or with the hydrogel, such as negatively affecting microorganisms or cells cultured in the hydrogel. Furthermore, it may in some applications not be possible to surface treat the plastic surface, to which a hydrogel is to be applied, such as due to mechanical restrictions, or the surface treatment negatively affects physical properties of the plastic surface, such as transparency.

Furthermore, additives have been suggested to modify the adhesive properties of hydrogels to strengthen the adhesion between the hydrogel and a plastic surface. Mao, Dynamics of agar-based gels in contact with solid surfaces: gelation, adhesion, drying and formulation, Université de Bordeaux, 2017 (https://tel.archives-ouvertes.fr/tel-01599047) concluded that anions enhanced the adhesion between agar gels and polystyrene surfaces and that the adhesion increased with salt concentration and was dependent on the nature of the anion species.

Addition of anion species to hydrogels as suggested above significantly improves adhesion between the hydrogel and plastic surfaces, in particular at higher anion concentrations. However, such anions present in the hydrogels have, as is shown herein, a negative effect on microorganisms or cells cultured in the anion-containing hydrogels.

There is, thus, a need for a technique to enhance adhesion between hydrogels and plastic surfaces that is not marred by shortcomings of the prior art techniques.

### SUMMARY

It is a general objective to provide a surface coating enhancing adhesion between hydrogels and plastic surfaces.

This and other objectives are met by embodiments of the present invention.

The present invention is defined in the independent claims. Further embodiments of the invention are defined in the dependent claims.

An aspect of the invention relates to a surface coating method. The method comprises heating a coating solution comprising a gelling agent, at least one sulfate salt and water to a temperature above the melting temperature of the gelling agent but below 100° C. The method also comprises applying the heated coating solution onto a plastic surface. The method further comprises drying the coating solution on the plastic surface to obtain a pre-coated plastic surface and applying a liquid hydrogel onto the pre-coated plastic surface. The method additionally comprises solidifying the liquid hydrogel to form a solid hydrogel adhering to the pre-coated plastic surface.

Another aspect of the invention relates to a plastic article. The plastic article comprises at least one pre-coated plastic surface obtainable by drying a heated coating solution applied onto at least one plastic surface of the plastic article and comprising a gelling agent, at least one sulfate salt and water. The heated coating solution is heated to a temperature above the melting temperature of the gelling agent but below 100° C. The plastic article also comprises a solid hydrogel on and adhering to the at least one pre-coated plastic surface.

A further aspect of the invention relates to a plastic article comprising at least one coated plastic surface obtainable by drying a heated coating solution applied onto at least one plastic surface of the plastic article. The heated coating solution comprises, preferably consists of, a gelling agent, at least one sulfate salt and water. The gelling agent is at a concentration selected within an interval of from 0.05 to 0.50% w/w. The at least one sulfate salt is at a concentration selected within an interval of from 0.25 to 2.00% w/w. The heated coating solution is heated to a temperature above the melting temperature of the gelling agent but below 100° C.

Yet another aspect of the invention relates to an adherence enhancing coating solution for a plastic surface. The adherence enhancing coating solution comprises, preferably consists of, a gelling agent, at least one sulfate salt and water. The gelling agent is at a concentration selected within an interval of from 0.05 to 0.50% w/w. The at least one sulfate salt is at a concentration selected within an interval of from 0.25 to 2.00% w/w.

The present invention solves adherence problems between hydrogels and plastic surfaces by pre-coating the plastic surfaces with a surface coating. The surface coating significantly improves the adherence of hydrogels to the pre-coated plastic surfaces and thereby reduces the risk of unintentional dislodging hydrogels from the plastic surfaces. This increase in adherence additionally does not require any modifications of the hydrogel material, which otherwise may have negative effects on microorganisms or cells cultured in and/or on the hydrogels.

### BRIEF DESCRIPTION OF DRAWINGS

The embodiments, together with further objects and advantages thereof, may best be understood by making reference to the following description taken together with the accompanying drawings, in which:

FIG. 1 is a flow chart illustrating a surface coating method according to an embodiment;

FIG. 2 schematically illustrates a coated plastic surface comprising a hydrogel according to an embodiment;

FIG. 3 schematically illustrates a Petri dish;

FIG. 4 schematically illustrates a multi-well plate as seen from above;

FIG. 5 is an illustration of a cassette assembly and its main components according to an embodiment;

FIG. 6 is a bottom view of a cassette assembly in connection with sample loading according to an embodiment;

FIG. 7 illustrates gel dislocation pressures for different surface coatings;

FIG. 8 illustrates gel dislocation pressures for different recipes and blood combinations;

FIG. 9 illustrates bacterial growth in Recipe 15 (9A) and Recipe 18 (9B); and

FIG. 10 illustrates mean cycle at which bacterial growth was detected in culture chambers of pre-coated sliders (CT) and uncoated sliders (not CT).

### DETAILED DESCRIPTION

The present invention generally relates to enhancing adherence between hydrogels and plastic surfaces, and in particular to a coating solution for coating plastic surfaces to enhance adherence between hydrogels and the coated plastic surface.

The present invention enhances the adhesion between hydrogels and plastic surfaces by applying a surface coating onto a plastic surface and then providing a hydrogel on the pre-coated plastic surface. The surface coating of the invention thereby enhances the adhesion between the hydrogel and the plastic surface. As a consequence, the risk of unintentional dislocation of the hydrogel from the plastic surface is significantly reduced as compared to directly providing the hydrogel onto the uncoated plastic surface. The enhancement in adhesion as achieved according to the present invention does, however, not come at the cost of negatively affecting microorganisms or cells cultured on or in the hydrogel, which is a problem associated with adding anions to hydrogels to improve their adhesion to plastic surfaces. In more detail, addition of anions into hydrogels to enhance their adhesion to plastic surfaces significantly reduced the growth rates of cells cultured in or on the modified hydrogels.

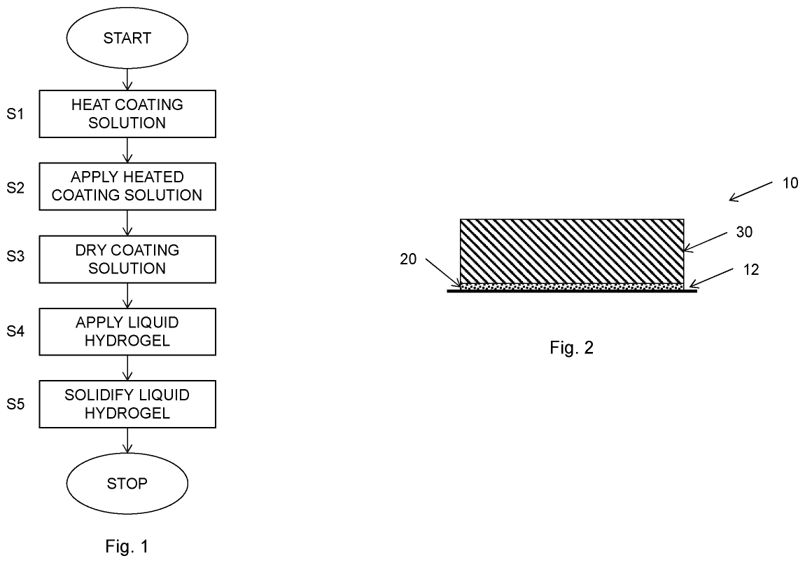

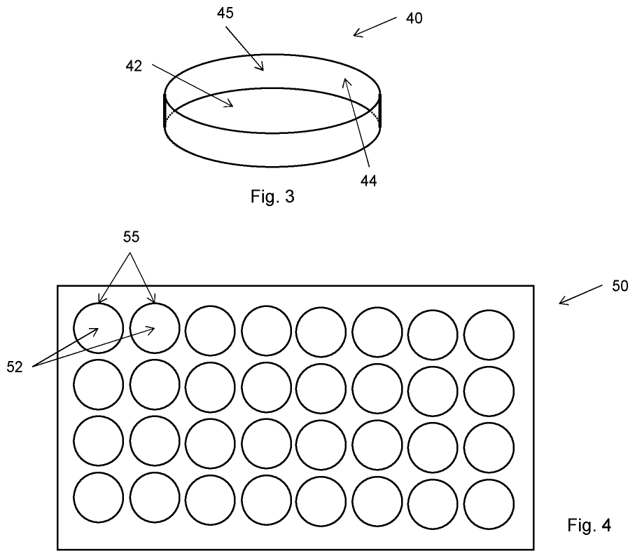

An aspect of the invention therefore relates to a surface coating method, see FIGS. 1 and 2. The method comprises heating, in step S1, a coating solution comprising a gelling agent, at least one sulfate salt and water to a temperature above the melting temperature of the gelling agent but below 100° C. The method also comprises applying the heated coating solution onto a plastic surface 12 in step S2. The method further comprises drying in step S3, the coating solution on the plastic surface 12 to obtain a pre-coated plastic surface. The method additionally comprises applying a liquid hydrogel onto the pre-coated plastic surface in step S4 and solidifying the liquid hydrogel to form a solid hydrogel 30 adhering to the pre-coated plastic surface.

The surface coating method of the invention thereby forms a pre-coated plastic surface with a surface coating 20 arranged between the plastic surface 12 and the solid hydrogel 30. This thin surface coating 20 thereby constitutes an interface between the plastic surface 12 and the solid hydrogel 30. The solid hydrogel 30 adheres stronger to the pre-coated plastic surface, i.e., to the surface coating 20, as compared to a corresponding non-coated plastic surface.

In an embodiment, the surface coating 20 has an average thickness on the plastic surface 12 equal to or less than 10 μm, preferably equal to or less than 5 μm, and more preferably equal to or less than 2.5 μm. In a particular embodiment, the surface coating 20 has an average thickness equal to or less than 1 μm.

Hydrogel as used herein refers to a crosslinked hydrophilic polymer that does not dissolve in water.

In an embodiment, the hydrogel is an agarose gel. Hence, in an embodiment, the solid hydrogel 30 adhering to the pre-coated plastic surface is preferably a solid agarose gel. Correspondingly, the liquid hydrogel applied in step S4 is preferably a liquid agarose gel. The embodiments are, however, not limited thereto but can be applied to other types of hydrogels. Illustrative, but non-limiting, examples of such hydrogels include agar, and polyethylene glycol. Currently preferred hydrogels include agarose gels and agar gels, in particular agarose gels.

An illustrative example of suitable agarose materials that could be used as hydrogel is ultra-low-gelling-temperature (ULGT) agarose. Other suitable materials include collagen materials, such as collagen I. Collagen I is well documented to support 3D cultures. Other gels that can be used include Engelbreth-Holm-Swarm (ECM) gels, such as Matrigel (BD Bioscience, Bedford, MA, USA) or hydrogels comprising a mixture of phenylalanine (Phe) dipeptide formed by solid-phase synthesis with a fluorenylmethoxycarbonyl (Fmoc) protector group on the N-terminus, and Fmoc-protected lysine (Lys) or solely phenylalanine. However, any type of biocompatible hydrogel could be used as long as the hydrogel can be applied in soluble form and cast or polymerized to form a solid hydrogel 30.

The plastic surface 12 could be a plastic surface of any plastic article, and in particular a plastic culture device, onto which a solid hydrogel 30 is to be provided and therefore to be adhered to a surface 12 of the plastic article, which is to be described further herein with reference to FIGS. 3 to 6.

The plastic material of the plastic article, or more correctly, of the plastic surface 12 could be any plastic material compatible with hydrogels and in particular compatible with microorganism or cell culturing. Illustrative, but non-limiting, examples of such plastic materials include poly(methyl methacrylate) (PMMA), polystyrene (PS), polyethylene (PE), polypropylene (PP), polyvinyl chloride (PVC), polyethylene terephthalate (PET), and polyurethane (PUR). Currently preferred plastic materials include PMMA and PS, in particular PMMA. Hence, in a particular embodiment, the plastic surface 12 is a PMMA surface.

In an embodiment, the gelling agent is made of a hydrophilic polymer of a hydrogel. Any gelling agent having a melting point below the boiling point of water, i.e., below 100° C., and that can be solidified on a plastic surface to obtain a pre-coated plastic surface can be used according to the embodiments. Illustrative, but non-limiting, examples of such gelling agents include agarose, agar and polyethylene glycol. A currently preferred gelling agent is selected among agarose and agar, and more preferably is agarose.

In a particular embodiment, the gelling agent is made of the same polymer as the hydrogel. Hence, in a currently preferred embodiment, the gelling agent and the hydrogel are made of the same hydrogel material, i.e., the same type of cross-linked hydrophilic polymer.

In an embodiment, the coating solution comprises the gelling agent at a concentration of at least 0.05% weight/weight (w/w). In a preferred embodiment, the coating solution comprises the gelling agent at a concentration selected within an interval of from 0.05 to 1.00% w/w, preferably at a concentration selected within an interval of from 0.05 to 0.50% w/w, and more preferably at a concentration selected within an interval of from 0.10 to 0.25% w/w. A currently preferred concentration is 0.125% w/w.

The at least one sulfate salt could be any sulfate salt comprising sulfate ions. In an embodiment, the at least one sulfate salt is selected from the group consisting of sodium sulfate (Na₂SO₄), potassium sulfate (K₂SO₄) and a mixture thereof. In an embodiment, a single sulfate salt is included in the coating solution, such as Na₂SO₄ or K₂SO₄. In another embodiment, a mixture or combination of two or more such sulfate salts is included in the coating solution, such as a mixture of Na₂SO₄ and K₂SO₄.

In an embodiment, the coating solution comprises the at least one sulfate salt at a concentration of at least 0.25% w/w. In a preferred embodiment, the coating solution comprises the at least one sulfate salt at a concentration selected within an interval of from 0.25 to 2.00% w/w, preferably at a concentration selected within an interval of from 0.25 to 1.00% w/w. A currently preferred concentration is 0.50% w/w.

In an embodiment, the coating solution consists of agarose powder at 0.125% w/w, Na₂SO₄ at 0.25% w/w, K₂SO₄ at 0.25% w/w and the balance being water, i.e., water at 99.375% w/w.

Experimental data as presented herein shows that the temperature of the coating solution during the applying step S2 in FIG. 1 has an effect on the adhesion properties of the solid hydrogel 30 applied and adhering onto the surface coating 20 on the plastic surface 12. Generally, the higher the temperature, the higher the adhesion. However, the coating solution is preferably not heated in step S1 too high to cause evaporation or deterioration of the coating solution. Hence, it is generally preferred to heat the coating solution in step S1 to a temperature above the melting temperature of the gelling agent in the coating solution but at a temperature below the boiling point of the coating solution. Hence, in a particular embodiment, the heating of the coating solution in step S1 is preferably conducted at a temperature above the melting temperature of the gelling agent but below about 100° C.

In an embodiment, step S1 in FIG. 1 comprises heating the coating solution to a temperature of at least 70° C. but below 100° C., preferably at a temperature selected within an interval of from 70° C. and 95° C., preferably within an interval of from 70° C. and 90° C., and more preferably within an interval of from 70° C. and 85° C. In a preferred embodiment, step S1 comprises heating the coating solution to a temperature selected within an interval of from 75° C. and 85° C., such as about 80° C.

The heating in step S1 is preferably performed to allow the gelling agent to melt in the coating solution and is preferably done to a temperature of the coating solution as described above. The heated coating solution is then applied in step S2 onto a plastic surface 12. The temperature of the coating solution as applied onto the plastic surface in step S2 may be the same temperature as the coating solution is heated to in step S1 or a lower temperature. For instance, the coating solution could be heated in step S1 to a temperature of about 80° C. The temperature of the coating solution when applied onto the plastic surface in step S2 could then be, for instance, about 70-80° C. Hence, the temperature of the coating solution applied in step S2 is preferably the same or a lower temperature as compared to the temperature of the coating solution during the heating step S1. In a preferred embodiment, the temperature of the coating solution as applied onto the plastic surface 12 is preferably above the melting point of the gelling agent.

The heated coating solution could be applied onto the plastic surface 12 according to various embodiments. The heated coating solution may, for instance, be sprayed onto the plastic surface 12 to form a thin surface coating 20. Alternatively, the heated coating solution could be poured onto the plastic surface 12 and then pouring off surplus coating solution to form the surface coating 20. A further alternative is to dip the plastic surface 12 into the heated coating solution.

The coating solution is then dried in step S3 on the plastic surface 12 to obtain a pre-coated plastic surface. The drying step S3 could be performed in an ambient environment. However, it is generally preferred to blow air onto the coated plastic surface 12 to obtain a dry surface coating 20 on the plastic surface 12. In such a case, pressurized gas, such as pressurized air, could be blown onto the coated plastic surface in step S3 to ensure that no liquid coating solution remains on the plastic surface 12 but rather a solid surface coating 20 is formed on the plastic surface 12.

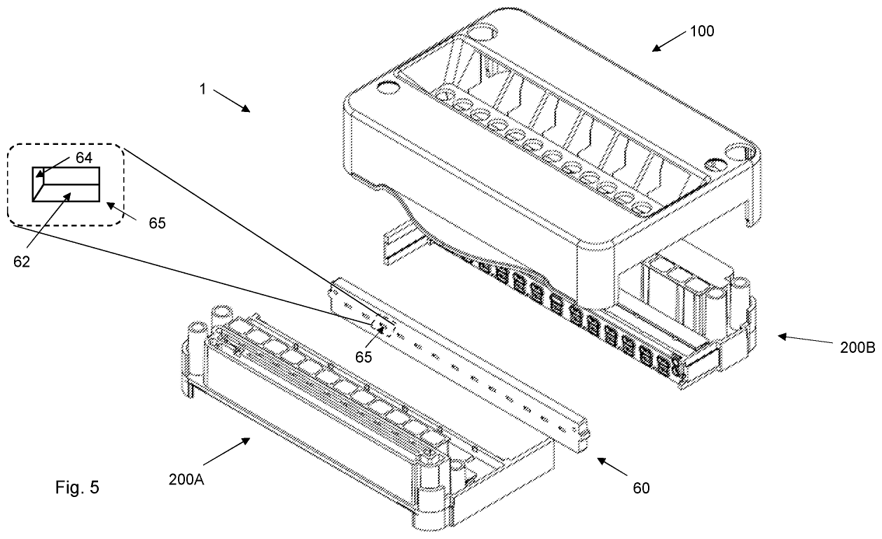

The plastic surface with the solid surface coating 20 formed in step S3 thereby constitutes a pre-coated plastic surface, onto which the liquid hydrogel is applied in step S4. The liquid hydrogel could be applied onto the pre-coated plastic surface in step S4 according to various embodiments and depending on the particular plastic surface 12. For instance, the liquid hydrogel could be poured into a pre-coated Petri dish 40, see FIG. 3, or into one or multiple pre-coated wells 55 of a multi-well plate 50, see FIG. 4. Hence, in such an embodiment, the liquid hydrogel is poured onto the pre-coated plastic surface in step S4. In another embodiment, the liquid hydrogel could be flown into a pre-coated culture chamber 65, see FIG. 5, in step S4. This step S4 of FIG. 1 could, thus, be performed according to various embodiments to apply the liquid hydrogel onto the pre-coated plastic surface.

In step S5 of FIG. 1, the liquid hydrogel is solidified to form a solid hydrogel 30 on and adhering to the pre-coated plastic surface. This means that the liquid hydrogel is cooled, cast and/or polymerized to form the solid hydrogel 30 that adheres to the pre-coated plastic surface.

As is schematically shown in FIG. 2, the average thickness of the solid hydrogel 30 is generally significantly larger than the average thickness of the surface coating 20. Thus, while the surface coating 20 preferably has an average thickness in the low μm range, or sub μm range, as mentioned above, the average thickness of the solid hydrogel 30 is typically in the sub mm, mm or cm range. Hence, in an embodiment, the average thickness of the solid hydrogel 30 is preferably at least 0.25 mm, preferably at least 0.5 mm, and more preferably at least 0.75 mm. These preferred average thicknesses are in particular suitable for solid hydrogels 30 present in culture chamber 65 of a slider 60 as shown in FIG. 5. Larger average thicknesses could be used for solid hydrogels 30 present in wells 45, 55 of Petri dishes 40 or multi-well plates 50, see FIGS. 3 and 5. In such a case, the average thickness of the solid hydrogel 30 could be at least 1 mm, preferably at least 2 mm, and more preferably at least 5 mm, or even larger, such as at least 7.5 mm, at least 10 mm, or at least 15 mm.

The adherence of the solid hydrogel 30 to the pre-coated plastic surface is significantly higher as compared to a corresponding adherence of the solid hydrogel 30 to a non-coated plastic surface, i.e., to the plastic surface 12 prior to applying the heated coating solution onto the plastic surface in step S2.

Another aspect relates to plastic article 10, 40, 50, 60, see FIGS. 2 to 6. The plastic article 10, 40, 50, 60 comprises at least one pre-coated plastic surface obtainable by drying a heated coating solution applied onto at least one plastic surface 12, 42, 44, 52, 62, 64 of the plastic article 10, 40, 50, 60 and comprising a gelling agent, at least one sulfate salt and water. The heated coating solution is heated to a temperature above the melting temperature of the gelling agent but below 100° C. The plastic article 10, 40, 50, 60 also comprises a solid hydrogel 30 on and adhering to the at least one pre-coated plastic surface.

In an embodiment, the pre-coated plastic surface comprises a surface coating 20 in the form of a gel coating 20 comprising, preferably consisting of, the gelling agent, the at least one sulfate salt and water.

In an embodiment, the surface coating 20 has an average thickness on the plastic surface 12 equal to or less than 10 μm, preferably equal to or less than 5 μm, and more preferably equal to or less than 2.5 μm. In a particular embodiment, the surface coating 20 has an average thickness equal to or less than 1 μm.

In an embodiment, the gelling agent is made of a hydrophilic polymer of a hydrogel. Illustrative, but non-limiting, examples of such gelling agents include agarose, agar, and polyethylene glycol. A currently preferred gelling agent is selected among agarose and agar, and more preferably is agarose.

In an embodiment, the solid hydrogel 30 is a solid agarose gel. The embodiments are, however, not limited thereto but can be applied to other types of hydrogels. Illustrative, but non-limiting, examples of such hydrogels include agar, and polyethylene glycol. Currently preferred hydrogels include agarose gels and agar gels, in particular agarose gels.

An illustrative example of a suitable agarose material for the hydrogel is ULGT agarose. Other suitable materials include collagen materials, such as collagen I. Collagen I is well documented to support 3D cultures. Other gels that can be used include ECM gels, such as Matrigel (BD Bioscience, Bedford, MA, USA) or hydrogels including a mixture of Phe dipeptide formed by solid-phase synthesis with a Fmoc protector group on the N-terminus, and Fmoc-protected Lys or solely phenylalanine. However, any type of biocompatible hydrogel could be used as long as the hydrogel material can be applied in soluble form and cast or polymerized to form a solid hydrogel.

The plastic material of the plastic article, or more correctly, of the plastic surface 12, 42, 44, 52, 62, 64 could be any plastic material compatible with solid hydrogels 30 and in particular compatible with cell culturing. Illustrative, but non-limiting, examples of such plastic materials include PMMA, PS, PE, PP, PVC, PET and PUR. Currently preferred plastic materials include PMMA and PS, in particular PMMA. In a particular embodiment, the plastic article 10, 40, 50, 60 is a PMMA article or is a plastic article 10, 40, 50, 60 comprising at least one PMMA surface 12, 42, 44, 52, 62, 64.

In an embodiment, the coating solution comprises the gelling agent at a concentration of at least 0.05% weight/weight w/w. In a preferred embodiment, the coating solution comprises the gelling agent at a concentration selected within an interval of from 0.05 to 1.00% w/w, preferably at a concentration selected within an interval of from 0.05 to 0.50% w/w, and more preferably at a concentration selected within an interval of from 0.10 to 0.25% w/w. A currently preferred concentration is 0.125% w/w.

The at least one sulfate salt could be any sulfate salt comprising sulfate ions. In an embodiment, the at least one sulfate salt is selected from the group consisting of Na₂SO₄, K₂SO₄ and a mixture thereof. In an embodiment, a single sulfate salt is included in the coating solution, such as Na₂SO₄ or K₂SO₄. In another embodiment, a mixture or combination of two or more such sulfate salts is included in the coating solution, such as Na₂SO₄ and K₂SO₄.

In an embodiment, the coating solution comprises the at least one sulfate salt at a concentration of at least 0.25% w/w. In a preferred embodiment, the coating solution comprises the at least one sulfate salt at a concentration selected within an interval of from 0.25 to 2.00% w/w, preferably at a concentration selected within an interval of from 0.25 to 1.00% w/w. A currently preferred concentration is 0.50% w/w.

In an embodiment, the coating solution consists of agarose powder at 0.125% w/w, Na₂SO₄ at 0.25% w/w, K₂SO₄ at 0.25% w/w and the balance being water, i.e., water at 99.375% w/w.

In an embodiment, the heated coating solution is heated to a temperature of at least 70° C. but below 100° C., preferably at a temperature selected within an interval of from 70° C. and 95° C., preferably within an interval of from 70° C. and 90° C., and more preferably within an interval of from 70° C. and 85° C. In a preferred embodiment, the heated coating solution is heated to a temperature selected within an interval of from 75° C. and 85° C., such as about 80° C.

In an embodiment, the average thickness of the solid hydrogel 30 is preferably at least 1 mm, preferably at least 2 mm, and more preferably at least 5 mm, or even larger, such as at least 7.5 mm, at least 10 mm, or at least 15 mm.

The plastic article 10, 40, 50, 60 could be any plastic article, onto which a hydrogel is to be applied. In a particular embodiment, the plastic article 10, 40, 50, 60 is a culturing device configured to culture microorganisms or cells on and/or in the solid hydrogel 30. The embodiments are, however, not limited to culturing devices or articles but could also be applied to other plastic article or devices where there is a need to attach a hydrogel onto a plastic surface of the plastic article or device.

In an embodiment, the plastic article 40, 50 is a plastic well plate 50, see FIG. 4, or dish 40, see FIG. 3, comprising at least one well 45, 55 having a well bottom 42, 52 and at least one well wall 44. In such an embodiment, at least a portion of the well bottom 42, 52 and/or of the well wall 44 is coated with the surface coating 20. Furthermore, the at least one well 45, 55 comprises a solid hydrogel 30 adhered to the pre-coated well bottom 42, 52 and/or pre-coated well wall 44.

FIG. 3 illustrates an example of the plastic article 40 in the form of a culture dish or Petri dish 40 comprising a well 45 having a well bottom 42 and a circular well wall 44. The liquid hydrogel is then added to the well 45 and solidified into a solid hydrogel 30 attached to the well bottom 42 and the circular well wall 44. In such a case, at least a portion of the well bottom 42, including the complete well bottom 42, could be coated with the surface coating 20 of the invention prior to adding the liquid hydrogel. Alternatively, or in addition, at least a portion of the circular well wall 44, including the complete well wall 44, could be coated with the surface coating 20 of the invention. For instance, the heated coating solution could be poured into the well 45 to form a surface coating 20 on the well bottom 44, or a portion thereof, and optionally onto at least a portion of the well wall 44 and then pouring off surplus heated coating solution.

FIG. 4 illustrates an example of the plastic article 50 in the form of a multi-well plate 50, such as a cell culture multi-well plate 50. The multi-well plate 50 then comprises a plurality of wells 55, typically arranged in an array or a matrix. In such a case, the heated coating solution could be applied to at least a portion of the well bottom 52 of all or a portion of the wells 55 and/or ono the well walls of all or a portion of the wells 55 to form the surface coating on the well bottom(s) 52 and/or well wall(s), or a portion thereof, prior to adding the liquid hydrogel.

The liquid hydrogel could be poured into the well 45 of the plastic article 40 of FIG. 3 or into one or more wells 55 of the multi-well plate 50 of FIG. 4 to fill up the complete volume of the well 45, 55 or merely a portion thereof, i.e., up to a portion of the height of circular wall(s) 44 of the well(s) 45, 55.

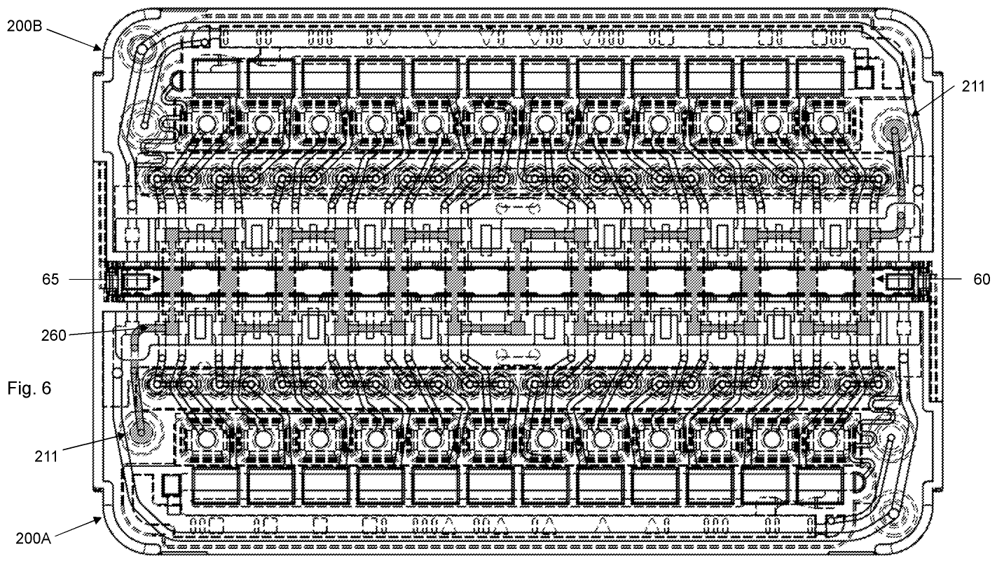

FIGS. 5 and 6 illustrate another example of a plastic article 60 in the form of a plastic culture device 60 comprising a plurality of culture chambers 65 comprising at least one chamber wall 62, 64 and in the form of through holes through the plastic culture device 60. In such an embodiment, the chamber walls 62, 64 of the plurality of culture chambers 65 are coated with the surface coating 20. The plurality of culture chambers 65 comprises a respective 3D culture matrix of a solid hydrogel 30 adhered to the at least one pre-coated chamber wall 62, 64.

In this embodiment, the plastic culture device 60 is a so-called slider 60 comprising a plurality of culture chambers 65 in the form of through holes through the slider 60. Such a slider 600 forms part of a cassette assembly 1 comprising interconnected cassette halves 200A, 200B with the slider 60 sandwiched between the cassette halves 200A, 200B, and a cover 100 to be attached to the cassette halves 200A, 200B. Such a cassette assembly is further described in WO 2020/204799.

FIG. 6 is a bottom view of the cassette assembly in FIG. 5 in connection with sample loading. In such a case, the at least one chamber walls 62, 64 of the culture chambers 65 have previously been coated with the heated surface coating. For instance, the slider 60 could be dipped in a bath of heated coating solution and then gently shaking the slider 60 from side to side in the bath to force out any air bubbles trapped in the culture chambers 65. The slider 60 may then be blow dried after the dip with pressurized air to ensure that no liquid remains are left on the slider 60. As a consequence, a surface coating 20 is formed in the culture chambers 65 on the at least one chamber walls 62, 64.

A sample in the form of a liquid hydrogel comprising microorganisms flows from an inlet port 211 in one of the cassette halves 200A, 200B through a channel system 260 and the culture chambers 65 in the slider 60 in a meander pattern to fill respective culture chamber 65 with the sample. The corresponding port 211 in the other cassette half 200B is preferably plugged with a filter, such as a filter plug, allowing air but not liquid to escape through the filter. This filter prevents any microorganisms, such as bacteria, in the sample from escaping through the port 211 and thereby contaminating the outside of the cassette assembly 1.

The cassette assembly 1 is then preferably placed inside a refrigerator to initiate and finish the gel reaction of the sample and thereby formation of solid 3D culture matrices (solid hydrogels 30) in the pre-coated culture chambers 65 of the slider 60. The cassette assembly 1 is then brought out from the refrigerator and is now ready for running an analysis of the response of microorganisms, such as bacteria, present in the sample to test agents, such as antibiotics, preloaded, such as in freeze-dried form, in reservoirs of one of the cassette halves 200A, 200B. At this point, the cassette assembly 1 can therefore be inserted into an analysis instrument of a fluidic system.

The pre-coating of the culture chambers 65 with the heated surface coating prevents or at least significantly reduces the risk of dislocation of the solid 3D culture matrices from the pre-coated culture chambers 65 during analysis when a fluid flow is applied onto the sides of the solid 3D culture matrices as disclosed in WO 2020/204799.

A further aspect of the invention relates to a plastic article 10, 40, 50, 60 comprising at least one pre-coated plastic surface obtainable by drying a heated coating solution applied onto at least one plastic surface 12, 42, 44, 52, 62, 64 of the plastic article 10, 40, 50, 60. The heated coating solution comprises, preferably consists of, a gelling agent, at least one sulfate salt and water. The gelling agent is at a concentration selected within an interval of from 0.05 to 0.50% w/w, preferably at a concentration selected within an interval of from 0.10 to 0.25% w/w, and more preferably at 0.125% w/w. The at least one sulfate salt is at a concentration selected within an interval of from 0.25 to 2.00% w/w, preferably at a concentration selected within an interval of from 0.25 to 1.00% w/w, and more preferably at 0.50% w/w. The heated coating solution is heated to a temperature above the melting temperature of the gelling agent but below 100° C.

Another aspect of the invention relates to an adherence enhancing coating solution for a plastic surface 12. The adherence enhancing coating solution comprises, preferably consists of, a gelling agent, at least one sulfate salt and water. The gelling agent is at a concentration selected within an interval of from 0.05 to 0.50% w/w, preferably at a concentration selected within an interval of from 0.10 to 0.25% w/w, and more preferably at 0.125% w/w. The at least one sulfate salt is at a concentration selected within an interval of from 0.25 to 2.00% w/w, preferably at a concentration selected within an interval of from 0.25 to 1.00% w/w, and more preferably at 0.50% w/w.

In an embodiment, the gelling agent is selected from the above-described gelling agents. In a particular embodiment, the gelling agent is agarose.

In an embodiment, the at least one sulfate salt is selected from the group consisting of Na₂SO₄, K₂SO₄ and a mixture thereof.

In an embodiment, the coating solution consists of agarose powder at 0.125% w/w, Na₂SO₄ at 0.25% w/w K₂SO₄ at 0.25% w/w and water at 99.375% w/w.

### EXAMPLES

### Example 1

This Example investigated problems associated with gel failures occurring in a cassette assembly as disclosed in WO 2020/204799. In more detail, a slider 60 of the cassette assembly 1 comprises culture chambers 65 in the form of through holes through the thickness of the slider 60, see FIG. 5. 3D culture matrices, such as in the form of agarose gels, will be formed in these culture chambers 65 and microorganism growth and response to test agents, such as antibiotics, can be monitored in these culture chambers 65.

Experimental results showed that there may be gel problems due to insufficient gel adhesion to the surfaces 62, 64 of the culture chambers 65 causing the gel plug (3D culture matrix) to loosen from the chamber walls 62, 64.

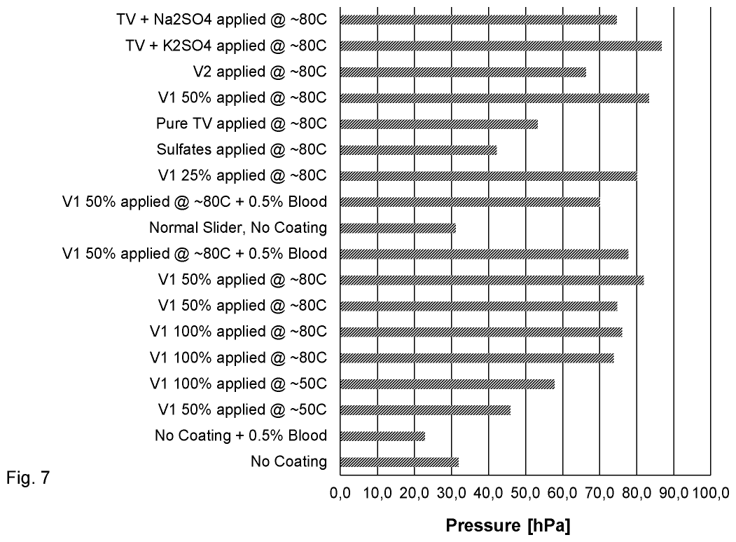

The process of coating sliders 60 with a hot solution of sulfate salts and agarose was explored. The developed coating procedure greatly increased the adhesion of the agarose gel to the culture chambers 65 as shown by the differences in gel dislocation pressure in FIG. 7.

The temperature, at which the coating was performed, played an important role in the performance of the surface coating as did the coating mixture itself, where both sulfate salts and a gelling agent (agarose) need to be used and removing either greatly reduced the performance of the surface coating.

### Coating and Slider Preparation

Six types of liquid coating solutions were tested during this Example, see Table 1. Coating solution V1 was additionally tested at two different temperatures and three different concentrations.

TABLE 1
List of initial coating solutions and ingredients

| Ingredients | Coating | Coating | Only | Pure | TV + | TV + |
|---|---|---|---|---|---|---|
| (w/w) | V1 | V2 | Sulfates | TV | Na₂SO₄ | K₂SO₄ |
| TopVision | 0.5% | 0.1% | — | 0.3% | 0.2% | 0.2% |
| Agarose | | | | | | |
| Na₂SO₄ Powder | 1.0% | 0.6% | 0.6% | — | 2.0% | — |
| K₂SO₄ Powder | 1.0% | 0.6% | 0.6% | — | — | 2.0% |

All prepared in purified water.

Each coating solution was applied by dipping a slider 60 (with protective film) in a bath of coating solution at 50-80° C. and then gently shaking each slider 60 from side to side in the bath to force out any air bubbles trapped in the culture chambers 65. Directly after the dip, each slider 60 was thoroughly blow dried with pressurized air, ensuring that no liquid remains were left on the slider 60.

### Gel

The 3D culture gel used in all tests to evaluate the slider coating was a commercially available gel consisting of 0.5% TopVision (TV) agarose dissolved in MH-II broth. Before injection into the slider 60, the refrigerated gel was heated to 80° C. and allowed to stabilize for 5-10 min followed by a cooling period down to around 35-40° C. Once the gel felt “body warm” it was injected into all 13 culture chambers 65 in the slider 60 using a pipette. The filled slider 60 was then placed in a refrigerator in a damp box at 4° C. for 8-10 min to allow the gel to harden before the actual tests began.

### Test Procedure

After 8-10 min in the refrigerator, the slider 60 with gel in the culture chambers 65 was placed with one of its narrow surfaces on a table on top of a paper towel. An adaptor was placed in front of a culture chamber 65 and clamped in place. A pressure meter was used for monitoring and logging the pressure on the gel surface of the culture chamber 65. A flow of 150 μl/min was started on a syringe pump followed by closing of the air valve once the liquid had passed the culture chamber 65.

Once the air valve was closed, the pressure started to slowly rise in the system, pushing on the gel sample in the culture chamber 65. Each experiment was stopped when either a sharp fall in pressure was registered on the pressure meter or when the gel sample became visibly dislocated and/or started leaking liquid. The adaptor was then moved to another culture chamber 65 on the same slider 60 and the procedure repeated 2-5 times on each slider 60.

### Results

The first interesting observation when using surface coated sliders instead of non-coated sliders was that the gel failure mode changed. In previous experiments with different gels, gel additives, and slider materials, the gel almost exclusively failed by getting completely ejected from the culture chamber. This happened suddenly and without warning. For the surface coated sliders the failure mode was different.

Instead of getting directly ejected, a tendril started to form on the high-pressure side of the gel that then gradually found its way to the other side over a period of seconds. Once the tendril reached the other side a cascading failure occurred that rapidly expanded the tendril and collapsed the gel, but even after this some parts of the gel remained in the culture chamber. This is interpreted as the gel structure itself cracking before detaching from the surface, whereas in previous experiments the gel had detached before it cracked.

The second observation from the gel dislocation pressure tests was that all tested surface coatings were better than none, see FIG. 7. Among all tested combinations, the ones containing both TV agarose and sulfate salts performed best. The temperature, at which the surface coating was applied, also seemed to affect the result, where hotter was better. Also of interest was the fact that the addition of 0.5% blood did not seem to affect the adhesion performance of the coated sliders that much as compared to Example 2, see description below, where blood had large negative effects.

V1 50% or 25% in FIG. 7 refers to a coating solution receipt having half or a quarter of the ingredients with the balance being water. For instance, coating V1 50%, which was selected as the best surface coating solution in FIG. 7, is shown in Table 2 below.

TABLE 2
V1 50 % coating solution recipe

| | First |
|---|---|
| | Evaluation |
| | Candidate |
| Ingredients (w/w) | (V1 50%) |
| Distilled H₂O | 98.75% |
| TopVision Agarose | 0.25% |
| Na₂SO₄ Powder | 0.50% |
| K₂SO₄ Powder | 0.50% |

### Example 2

This Example involved modifying the standard TopVision agarose gel by the addition of sulfate salts and monitoring the changes in the adhesion properties of the gel.

The chosen additives, however, negatively affected bacterial growth when bacteria were set to grow in the gel. Furthermore, addition of blood to the gel significantly reduced the gel adhesion.

The test procedure was performed as in Example 1 but with the difference that no surface coating of the slider was performed. In clear contrast, different gel recipes were tested, in which additives were added to the gel.

### Gel Development

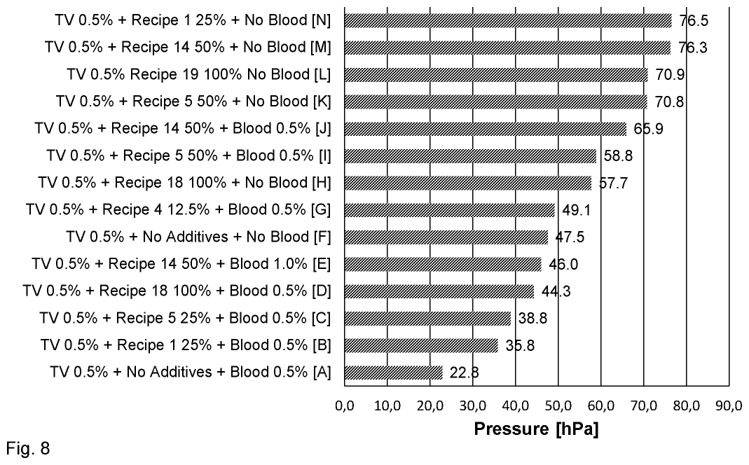

In total, 19 different controlled gel recipes were developed. A summary of the additive ion concentrations of the most promising recipes are shown in Table 3 and their corresponding adhesion performance is shown in FIG. 8.

TABLE 3
Additive ion concentrations for different recipes

| | Recipe 1 | Recipe 5 | Recipe 14 | Recipe 15 | Recipe 18 | Recipe 19 |
|---|---|---|---|---|---|---|
| Ion [mg/g] | 25% conc. | 50% conc. | 50% conc. | 100% conc. | 100% conc. | 100% conc. |
| SO₄ | 9.30 | 10.15 | 16.00 | 16.00 | 7.21 | 9.61 |
| Na | 4.45 | 4.86 | 4.82 | 4.82 | 2.30 | 2.30 |
| K | 5.61 | 5.19 | 4.82 | 4.82 | 1.96 | 3.91 |
| Ca | — | 0.036 | 0.036 | 0.036 | — | — |
| Cl | 5.09 | 4.77 | 0.064 | 0.064 | — | — |
| Total % | 2.45 | 2.50 | 2.57 | 2.57 | 1.15 | 1.58 |
| w/w | | | | | | |

As shown in FIG. 8, the standard gel with no added additives or blood could resist about 47.5 hPa [F] before being dislocated. Adding 0.5% horse blood to this standard mixture caused this value to fall to, on average, 22.8 hPa [A], a reduction of 52%.

Adding Recipe 1 at 25% concentration to the gel increased the adhesion to, on average, 76.5 hPa [N], a 61% increase over the standard gel [F]. When adding 0.5% blood to this sample, the adhesion fell to 35.8 hPa [B], a reduction of 53% compared to the no blood case, but still 57% stronger than the unmodified gel [A].

Pure gel mixed with Recipe 5 at 50% concentration had an initial adhesion of 70.8 hPa [K], falling to 58.8 hPa [I] when adding 0.5% blood, a reduction of 17% but still 158% better than the unmodified gel [A].

Recipe 14 at 50% concentration showed a similar initial adhesion to that of Recipe 1 of 76.3 hPa [M]. Adding 0.5% blood to this Recipe 14 mixture reduced this value to 65.9 hPa [J], a reduction of only 14% and 189% better than the unmodified gel [A]. Adding 1% blood to the same sample further reduced the adhesion to 46.0 hPa [F], a 40% reduction in adhesion compared to its pure form but still maintaining a 102% lead over the unmodified gel [A].

Moving on to gels with recipes adjusted to be useful at 100% concentration, pure gel based on Recipe 18 could withstand pressures around 57.7 hPa [J] and dropping to 44.3 hPa [D] if 0.5% blood was added, a reduction of 23%, but still 94% above that of the unmodified gel [A].

Pure gel based on Recipe 19 could withstand pressures around 70.9 hPa [L]. This recipe was never tested with blood.

Judging from these results it seemed that increased ion content corresponded to an increase in gel adhesion similar to the conclusions in Mao, Dynamics of agar-based gels in contact with solid surfaces: gelation, adhesion, drying and formulation, Université de Bordeaux, 2017 (https://tel.archives-ouvertes.fr/tel-01599047). Furthermore, the magnitude of the increase also depended on the ratio of ions, as seen if one compared Recipe 5 at 50% and Recipe 19 at 100%, where both had almost identical adhesion in their pure form but a 58% difference in ion concentration. Adding blood decreased the adhesion of all tested gel recipes.

### Biological Evaluation

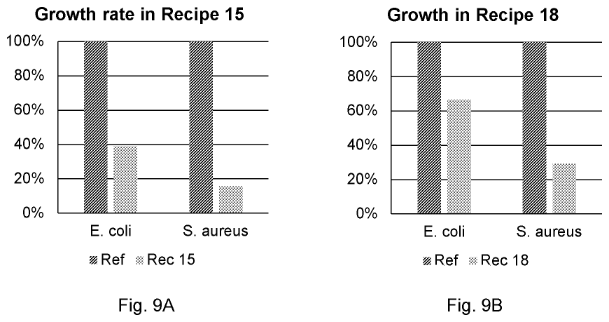

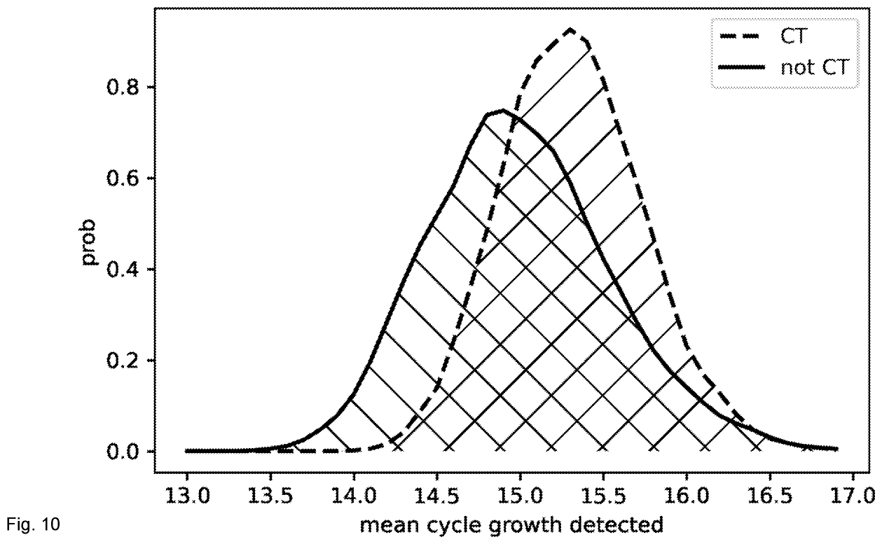

FIGS. 9A and 9B show the relative growth rates for two bacterial species (Escherichia coli and Staphylococcus aureus) in gels according to Recipe 15 or Recipe 18. Both recipes inhibited bacterial growth and increasing ion concentration (Recipe 15) gave a stronger inhibiting effect.

### Example 3

This example investigated cell culturing in gel plugs (3D culture matrix) in culture chambers 65 of a slider 60 as shown in FIGS. 5 and 6 and in a cassette assembly as disclosed in WO 2020/204799.

3D culture gels according to Example 1 but comprising Klebsiella pneumoniae (ATCC 700603) cells were injected into culture chambers 65 of nineteen sliders 60 pre-coated with a surface coating consisting of TopVision agarose powder at 0.125% w/w, Na₂SO₄ at 0.25% w/w, K₂SO₄ at 0.25% w/w and balance being water and into ten uncoated sliders 60. The culture chambers 65 were monitored to detect bacterial growth. The monitoring cycle, at which bacterial growth was detected in 95% of the culture chamber 65 during three consecutive monitoring cycles was noted. The results are presented in FIG. 10 and indicate that the mean cycle at which bacterial growth was detected in culture chambers 65 of pre-coated sliders 60 (CT) was 15.2 cycles, whereas the corresponding mean cycle at which bacterial growth was detected in culture chambers 65 of uncoated sliders 60 (not CT) was 14.9. Hence, there was no significant difference in bacterial growth between pre-coated and uncoated sliders 60. Accordingly, pre-coating the culture chambers 65 to enhance the adhesive properties of the 3D culture gels to the culture chambers 65 did not negatively affect bacterial growth in the 3D culture gels.

### Conclusions from Example 1 to 3

Pre-coating the slider with a surface coating had several significant advantages as compared to adding ion additives directly into the gel. These advantages included no significant loss in adhesive properties when adding blood, generally enhanced adhesive properties, and no detrimental effects on bacterial growth.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible.

1.-30. (canceled)

31. A surface coating method comprising:
heating a coating solution comprising a gelling agent, at least one sulfate salt and water to a temperature above the melting temperature of the gelling agent but below 100° C.;
applying the heated coating solution onto a plastic surface;
drying the coating solution on the plastic surface to obtain a pre-coated plastic surface;
applying a liquid hydrogel onto the pre-coated plastic surface; and
solidifying the liquid hydrogel to form a solid hydrogel adhering to the pre-coated plastic surface.

32. The method according to claim 31, wherein the solid hydrogel is a solid agarose gel.

33. The method according to claim 31, wherein the plastic surface is a poly(methyl methacrylate) (PMMA) surface.

34. The method according to claim 31, wherein the gelling agent is agarose.

35. The method according to claim 31, wherein the coating solution comprises the gelling agent at a concentration of at least 0.05% w/w.

36. The method according to claim 35, wherein the coating solution comprises the gelling agent at a concentration selected within an interval of from 0.05 to 1.00% w/w.

37. The method according to claim 36, wherein the coating solution comprises the gelling agent at a concentration selected within an interval of from 0.10 to 0.25% w/w.

38. The method according to claim 37, wherein the at least one sulfate salt is selected from the group consisting of Na₂SO₄, K₂SO₄ and a mixture thereof.

39. The method according to claim 31, wherein the coating solution comprises the at least one sulfate salt at a concentration of at least 0.25% w/w.

40. The method according to claim 39, wherein the coating solution comprises the at least one sulfate salt at a concentration selected within an interval of from 0.25 to 2.00% w/w.

41. The method according to claim 40, wherein the coating solution comprises the at least one sulfate salt at a concentration selected within an interval of from 0.25 to 1.00% w/w.

42. The method according to claim 31, wherein the coating solution consists of:
agarose powder at 0.125% w/w;
Na₂SO₄ at 0.25% w/w;
K₂SO₄ at 0.25% w/w; and
water at 99.375% w/w.

43. The method according to claim 31, wherein heating the coating solution comprises heating the coating solution to a temperature of at least 70° C. but below 100° C.

44. The method according to claim 43, wherein heating the coating solution comprises heating the coating solution to a temperature selected within an interval of from 70° C. and 85° C.

45. The method according to claim 31, wherein drying the coating solution comprises blowing air onto the coated plastic surface to obtain a dry surface coating on the plastic surface.

46. A plastic article comprising:
at least one pre-coated plastic surface obtainable by drying a heated coating solution applied onto at least one plastic surface of the plastic article and comprising a gelling agent, at least one sulfate salt and water, wherein the heated coating solution is heated to a temperature above the melting temperature of the gelling agent but below 100° C.; and
a solid hydrogel on and adhering to the at least one pre-coated plastic surface.

47. The plastic article according to claim 46, wherein the pre-coated plastic surface comprises a surface coating in the form of gel coating comprising the gelling agent, the at least one sulfate salt and water.

48. The plastic article according to claim 47, wherein the surface coating has an average thickness on the plastic surface equal to or less than 10 μm.

49. The plastic article according to claim 48, wherein the surface coating has an average thickness on the plastic surface equal to or less than 2.5 μm.

50. The plastic article according to claim 46, wherein the gelling agent is agarose.

51. The plastic article according to claim 46, wherein the solid hydrogel is a solid agarose gel.

52. The plastic article according to claim 46, wherein the plastic article is a poly(methyl methacrylate) (PMMA) article or is a plastic article comprising at least one PMMA surface.

53. The plastic article according to claim 46, wherein the coating solution comprises the gelling agent at a concentration of at least 0.05% w/w.

54. The plastic article according to claim 53, wherein the coating solution comprises the gelling agent at a concentration selected within an interval of from 0.05 to 1.00% w/w.

55. The plastic article according to claim 54, wherein the coating solution comprises the gelling agent at a concentration selected within an interval of from 0.10 to 0.25% w/w.

56. The plastic article according to claim 46, wherein the at least one sulfate salt is selected from the group consisting of Na₂SO₄, K₂SO₄ and a mixture thereof.

57. The plastic article according to claim 46, wherein the coating solution comprises the at least one sulfate salt at a concentration of at least 0.25% w/w.

58. The plastic article according to claim 57, wherein the coating solution comprises the at least one sulfate salt at a concentration selected within an interval of from 0.25 to 2.00% w/w.

59. The plastic article according to claim 58, wherein the coating solution comprises the at least one sulfate salt at a concentration selected within an interval of from 0.25 to 1.00% w/w.

60. The plastic article according to claim 46, wherein the coating solution consists of:
agarose powder at 0.125% w/w;
Na₂SO₄ at 0.25% w/w;
K₂SO₄ at 0.25% w/w;
water at 99.375% w/w.

61. The plastic article according to claim 46, wherein the heated coating solution is heated to a temperature of at least 70° C. but below 100° C.

62. The plastic article according to claim 46, wherein the heated coating solution is heated to a temperature selected within an interval of from 70° C. and 85° C.

63. The plastic article according to claim 46, wherein
the plastic article is a plastic culture device comprising a plurality of culture chambers comprising at least one chamber wall and in the form of through holes through the plastic culture device;
the chamber walls of the plurality of culture chambers are pre-coated with the surface coating; and
the plurality of culture chambers comprises a respective three-dimensional culture matrix of a solid hydrogel adhered to the at least one pre-coated chamber wall.

64. The plastic article according to claim 63, wherein the solid hydrogel has an average thickness equal to or larger than 0.25 mm.

65. The plastic article according to claim 46, wherein
the plastic article is a plastic well plate or dish comprising at least one well having a well and at least one well wall;
at least a portion of the well bottom and/or of the well wall is pre-coated with the surface coating; and
the at least one well comprises a solid hydrogel adhered to the pre-coated well bottom and/or coated well wall.

66. The plastic article according to claim 65, wherein the solid hydrogel has an average thickness equal to or larger than 1 mm.

67. A plastic article comprising at least one pre-coated plastic surface obtainable by drying a heated coating solution applied onto at least one plastic surface of the plastic article and comprising, preferably consisting of:
a gelling agent at a concentration selected within an interval of from 0.05 to 0.50% w/w;
at least one sulfate salt at a concentration selected within an interval of from 0.25 to 2.00% w/w; and
water, wherein the heated coating solution is heated to a temperature above the melting temperature of the gelling agent but below 100° C.

68. An adherence enhancing coating solution for a plastic surface comprising:
at least one sulfate salt at a concentration selected within an interval of from 0.25 to 2.00% w/w;
a gelling agent at a concentration selected within an interval of from 0.05 to 0.50% w/w; and
water.

69. The coating solution according to claim 68, wherein the gelling agent is agarose.

70. The coating solution according to claim 68, wherein the at least one sulfate salt is selected from the group consisting of Na₂SO₄, K₂SO₄ and a mixture thereof.

71. The coating solution according to claim 68, wherein the coating solution consists of:
agarose powder at 0.125% w/w;
Na₂SO₄ at 0.25% w/w;
K₂SO₄ at 0.25% w/w; and
water at 99.375% w/w.
